(19) United States
(12) Patent Application Publication
(10) Pub. No.: US 2008/0268069 A1
(43) Pub. Date: Oct. 30, 2008

(54) Mixture having effect of improving insulin resistance, effect of suppressing body weight gain, and effect of preventing and improving fatty liver

(75) Inventors: Tsutomu Yae, Tokyo (JP); Orie Yoshinari, Minami-Saitama-gun (JP)

Correspondence Address: PRICE HENEVELD COOPER DEWITT & LITTON, LLP, 695 KENMOOR, S.E., P O BOX 2567, GRAND RAPIDS, MI, 49501, US

(73) Assignee: Ryusendo Co., Ltd.

(21) Appl. No.: 11/789,594

(22) Filed: Apr. 25, 2007

(51) Int. Cl. A61K 33/24; A61K 36/8962; A61P 3/00

(52) U.S. Cl. 424/655; 424/754

(57) ABSTRACT

The specification discloses a mixture obtained by mixing trivalent chromium and an onion extract, which has the effect of improving insulin resistance, suppressing body weight and preventing and improving fatty liver. Preferably the trivalent chromium is in a form of trivalent chromium-nicotinic acid complex.

### BACKGROUND OF THE INVENTION

1. Field of the Invention

The present invention relates to a mixture obtained by mixing chromium and an onion extract, having an effect of improving insulin resistance, an effect of suppressing body weight gain and an effect of preventing and improving fatty liver that can appear in a short period of time. Particularly, the present invention relates to a mixture in which the chromium is in the form of a trivalent chromium-nicotinic acid complex.

2. Description of the Related Art

Studies on diabetes have been previously performed in Japan and other countries, and the present inventors have also carried out studies on diabetes, particularly on the improvement of insulin resistance.

Insulin resistance is a condition in which the action of insulin as the only blood glucose-lowering hormone is not exerted and glucose uptake is suppressed, for example, in the liver, the skeletal muscles, and adipocytes. Naturally, the body secretes insulin from β-cells in the islets of Langerhans for controlling blood glucose when a blood glucose level is elevated by diet, and insulin binds to insulin receptors in the liver, skeletal muscles and adipocytes to promote uptake of glucose. However, even if a sufficient amount of insulin is secreted from β-cells does, the receptors cannot sense insulin and glucose uptake does not occur when there is an impairment in insulin receptors or subsequent pathways in organs. In other words, reduced insulin sensitivity is called insulin resistance, with which the occurrence of diabetes, hyperlipemia, and hypertension are facilitated, and arteriosclerosis is developed.

Obesity (increased body weight) is attributed to an increased proportion of adipocytes, and adipocytes that have become huge are known to secrete insulin resistance-producing substances such as free fatty acids, TNF-α, and resistin. Thus, obesity is known to be associated with insulin resistance.

Obesity due to accumulated visceral fat inevitably occurring by recent full feeding and machine civilization, impacts of a motorized society, and genetic backgrounds offers a key to life-style related diseases (metabolic syndromes). The degree of obesity is judged using BMI (Body Mass Index) and calculated according to body weight (kg)/(height (m))². The Obesity Diagnosis Criteria Examination Committee of the Japan Society for the Study of Obesity (2000) has prescribed that a BMI of 25 or more indicates obesity.

The national nutrition survey conducted by the Ministry of Health and Welfare in 2003 shows that the ratio of male obese individuals increased in all age-groups compared to that in 1983. A BMI of 25 or more with a waist circumference of 85 cm or more in men and 90 cm or more in women was defined as a suspicion of upper-body obesity, which when surveyed, accounted for 24.9% of cases in men and 13.8% in women.

As just described, obesity, i.e. increased body weight, has become an important problem in recent years.

Accordingly, the present inventors have conducted intensive studies on the improvement of insulin resistance and the inhibition of obesity, i.e., the suppression of body weight gain.

The present inventors have first focused attention on onion. Onion has been variously studied and is known to have an antidiabetic effect (see, for example, Japanese Patent Laid-Open No. 2005-247733 and Japanese Patent Laid-Open No. 10-077232), a lipid-lowering effect (see, for example, Japanese Patent Laid-Open No. 2003-192604), and the like.

Attention has been then focused on chromium since chromium is known to aid the action of insulin to maintain normal blood glucose levels.

Trivalent chromium sometimes causes skin inflammation when parenterally administered in large amounts but has no toxicity when orally administered in small amounts. Hexavalent chromium is known to have strong toxicity.

Chromium has had a problem that it is less easily absorbed into the body; however, a trivalent chromium-nicotinic acid complex, which has been invented by the William Seroy group and patented under Japanese Patent No. 3014109, has been found to be very easily absorbed into the body (see, for example, Olin K L, Steams D M, Armstrong W H, Keen C L, Comparative Retention/Absorption of 51Chromium (51Cr) from 51Cr Chloride, 51Cr Nicotinate and 51Cr Picolinate in a Rat Model, Trade Elements, and Electrolytes, 11: 182-186, 1994.).

However, with regard to onion no documents suggest the suppression of body weight gain or describe experimental examples thereof. Insulin resistance is suggested to be potentially improved by administering onion for a long period of approximately 3 months or more (see, for example, Japanese Patent Laid-Open No. 2003-192604), but there are no documents in which clear-cut experiments or demonstrations showing the improvement have been made. In addition, the synergistic effect thereof with chromium is not described in any document.

### SUMMARY OF THE INVENTION

The present inventors have believed that mixing an onion extract with trivalent chromium, particularly a trivalent chromium-nicotinic acid complex may enable a synergistic effect on improving insulin resistance and suppressing body weight gain to be expected.

Accordingly, as a result of intensive studies, it has been found that a mixture of an onion extract and trivalent chromium, particularly a trivalent chromium-nicotinic acid complex has marked effects in improving insulin resistance and suppressing body weight gain. Further, it has been also found that the mixture has a pronounced effect in preventing and improving fatty liver.

Thus, an object of the present invention is to provide a mixture having effects of improving insulin resistance and suppressing body weight gain (obesity) and an effect of preventing and improving fatty liver.

For achieving the above-described object, the mixture having an effect of improving insulin resistance, an effect of suppressing body weight gain, and an effect of preventing and improving fatty liver is obtained by mixing trivalent chromium and an onion extract.

The trivalent chromium is preferably in the form of a trivalent chromium-nicotinic acid complex.

The structural formula of the trivalent chromium-nicotinic acid complex is as follows.

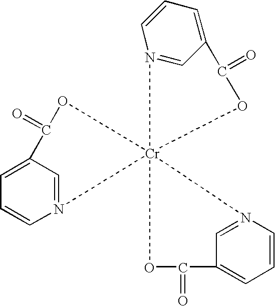

According to the present invention, there can be provided a mixture having an effect of improving insulin resistance, an effect of suppressing body weight gain (obesity) and an effect of preventing and improving fatty liver which can appear in a short period of the order of 2 to 4 weeks.

### DETAILED DESCRIPTION OF THE PREFERRED EMBODIMENT

### Example

A production example is as follows.

Ten kilograms of onions from Hokkaido, Japan were washed twice with water using a washer and immediately placed in an oven, followed by heat-boiling at 100° C. The resultant boiled onions were crushed with a feather mill and subjected to juice extraction using a juice extractor to provide 9.5 kg of a juice extract. The 9.5 kg of juice extract is concentrated to 0.9 kg of concentrated extract in a centrifugal thin film type vacuum concentrator at a temperature of 92° C. and a pressure of 15 mmHg (for an evaporating temperature of 42° C.). The component distilled in the vacuum concentration was put into an activated carbon layer for adsorption. The adsorbate was eluted with ethanol, followed by removing ethanol from the resultant solution by evaporation to provide 1.5 g of the remaining component. This component was uniformly added to the concentrated extract previously obtained to provide 0.9 kg of an onion extract. Dextrin was added to the resultant extract, which was then spray-dried to make a powder.

Nicotinic acid is added to water for dissolution, to which trivalent chromium chloride is then added, followed by stirring the solution. The stirring provides a precipitate of a trivalent chromium-nicotinic acid complex. The precipitate is finely crushed using a crusher and dried to make a powder.

The onion powder and powder of trivalent chromium or the trivalent chromium-nicotinic acid complex thus obtained are mixed using a mixer to provide a mixture.

The mixing ratio is preferably about 1.00 to 0.01% by weight of the powder of the trivalent chromium-nicotinic acid complex having a chromium content of 10% by weight to about 99.00 to 99.99% by weight of the onion extract powder.

The method for producing the onion extract powder and the method for producing the powder of trivalent chromium or the trivalent chromium-nicotinic acid complex are methods which have been conventionally used. The mixture of these powders can be further made in the form of a tablet, a capsule, or the like using a commonly used method to use the formulation as a health food, or can be, as needed, subjected to further addition of pharmaceutically acceptable additives (for example, an excipient, a surfactant, and the like) to use the resultant mixture as a pharmaceutical preparation.

An example is described below.

Purpose

Study of a synergistic effect of a trivalent chromium-nicotinic acid complex and an onion extract on the improvement of obesity and insulin resistance

Method

The Zucker fa/fa rat is polyphagous and used as an obesity model and also as a hyperinsulinemia or insulin resistance model. Accordingly, the rat was used in this study to examine a synergistic effect of a trivalent chromium-nicotinic acid complex, which is believed to be a glucose tolerance factor, and an onion extract.

### Experimental Animals

Eight-week old Zucker +/+(Non-obesity model)

Eight-week old Zucker fa/fa (Obesity model)

### Groups

Control (Con) +/+Basic diet

Vehicle (V) fa/fa Basic diet

Chromium (III) (Cr) fa/fa Basic diet+5 ppm

### Trivalent Chromium-Nicotinic Acid Complex

Onion (O) fa/fa Basic diet+5% Onion

Cr+O (CrO) fa/fa Basic diet+5 ppm Trivalent

chromium-nicotinic acid complex+5% Onion
Six animals per group (feed and water are given ad libritum)
Feeding period: 28 days (8 to 11 weeks old)
The content of chromium in the trivalent chromium-nicotinic acid complex is 10% by weight.

Diet composition

| (%) | Con | V | Cr | O | CrO |
|---|---|---|---|---|---|
| Casein | 20.0 | 20.0 | 20.0 | 15.0 | 15.0 |
| α-Cornstarch | 43.7 | 43.7 | 43.7 | 43.7 | 43.7 |
| Sucrose | 21.8 | 21.8 | 21.8 | 21.8 | 21.8 |
| Corn oil | 5.0 | 5.0 | 5.0 | 5.0 | 5.0 |
| Cellulose | 5.0 | 5.0 | 5.0 | 5.0 | 5.0 |
| Vitamin mixture | 3.5 | 3.5 | 3.5 | 3.5 | 3.5 |
| Mineral mixture | 1.0 | 1.0 | 1.0 | 1.0 | 1.0 |
| Trivalent chromium•nicotinic | — | — | 5 ppm | — | 5 ppm |
| acid complex | | | | | |
| Onion | — | — | — | 5.0 | 5.0 |

Measurement Items

Dietary intake, body weight gain, liver weight, kidney weight, peritesticular fat weight, glucose tolerance test, blood glucose level, serum insulin, total serum cholesterol, serum triglyceride, serum free fatty acid, and serum leptin.

Statistical Analysis

Significant tests were carried out using the t test. Two-tailed tests were used for the V group, with significance expressed at p<0.05 (*) and p<0.01 (**).

Results

Body weight gain and total dietary intake

When body weight and dietary intake were measured during a feeding period of 28 days, no difference in total dietary intake was observed among the V, Cr, 0, and CrO groups without the Con group. However, a significant difference in the suppression of body weight gain was noted in the CrO group when compared to the V group. In contrast, no significant difference in the suppression was observed in the Cr and 0 groups when compared to the V group.

More specifically, the increase in body weight tended to be suppressed only in the CrO group compared to that in the V group and significantly depressed in the former group at the 21st day and later.

This showed that the mixture of the present invention had the effect of suppressing body weight gain (the effect of preventing obesity) despite that neither the trivalent chromium-nicotinic acid complex alone nor the onion extract alone had the effect. In addition, the mixture was noted to have the effect of suppressing body weight gain in a short period of time.

Weight of Liver, Kidney, and Peritesticular Fat

A significant difference in liver weight was observed in the CrO group when compared to the V group. In contrast, no significant difference therein was observed in the Cr and O groups when compared to the V group. The liver of the fa/fa rats was an apparent fat liver compared to that of the rats of the Con group.

This showed that the mixture of the trivalent chromium-nicotinic acid complex and the onion extract had the prominent advantage of having an effect of preventing and improving fat liver despite that neither the trivalent chromium-nicotinic acid complex alone nor the onion extract alone had the effect.

In this respect, the cause of the above-described suppression of body weight gain is unknown, but there may be a possibility that fat deposition in the organ is reflected in the body weight because the suppression was significantly observed only in the CrO group (significance level: p<0.05). It was also suggested that the effect was organ-specific because no significant difference in the weight of kidney and peritesticular fat was observed in the Cr, 0 and CrO groups when compared to the V group.

Glucose Tolerance Test

Glucose tolerance tests were carried out in each group of rats at the 2nd week (9-week old) and 4th (11-week old) week after the start of experiment. After 16 hours of fasting, each group of rats was forcedly allowed to ingest 2 g/kg body weight of D-glucose, followed by measuring blood glucose level with time. As a result, the level in the CrO group showed a significantly suppressed increase 15 minutes after the administration of D-glucose and was significantly decreased at 120 minutes, compared to that in the V group. In contrast, no significant difference in the level was observed in the Cr and O groups when compared to the V group.

Serum Insulin and Blood Glucose Level

At autopsy, serum insulin and blood glucose level were measured. As a result, no significant difference in serum insulin content was observed in the Cr, O and CrO groups when compared to the V group. A significant difference in blood glucose level was noted in the CrO group when compared to the V group. In contrast, no significant difference in the level was observed in the Cr and O groups when compared to the V group.

These data showed that no difference in serum insulin content was noted in the CrO group when compared to the Cr, O and V groups despite the prominent effect observed in the CrO group in the glucose tolerance test, i.e. that the significant difference in blood glucose level was found in the CrO group when compared to the V group, at 15 minutes and 120 minutes at the 2nd week of feeding; in the CrO group at the 4th week, there were noted the significantly suppressed increase in the level at 30 minutes and the decrease of the level to the blood glucose level in the non-obese +/+ rats at 120 minutes.

From these results, it is probable that the combined use of the trivalent chromium-nicotinic acid complex and the onion extract improved insulin sensitivity and promoted glucose incorporation without affecting insulin-secreting capacity.

In other words, the mixture of the trivalent chromium-nicotinic acid complex and the onion extract was observed to have a prominent effect of improving insulin resistance in a short period of 2 to 4 weeks despite that neither the trivalent chromium-nicotinic acid complex alone nor the onion extract alone had the effect of improving insulin resistance.

Total Serum Cholesterol (TC), Serum Triglyceride (TG), Serum Free Fatty Acid (FFA), and Leptin

No significant difference in TC, FFA, and leptin was observed in the Cr, O and CrO groups when compared to the V group. A significant difference in TG was noted in the CrO group when compared to the V group. In contrast, no significant difference in TG was observed in the Cr and O groups when compared to the V group. A tendency of reduction, but not a significant difference, in FFA was noted in the CrO group when compared to the V group. The Zucker fa/fa rat is known to have a high serum leptin content due to leptin receptor mutation, but the rats showed no change in the leptin content in the present experiment.

No difference in serum TC was present among the groups of fa/fa rats, but a tendency of reduction in TG in the Cr and O groups and a significant reduction therein in the CrO group were observed when compared to the V group. A tendency of reduction, but not a significant difference, in FFA was also noted in the CrO group when compared to the V group. An insufficient insulin action depresses lipoprotein lipase activity, impairs chylomicron and VLDL catabolism, and increases blood TG levels, resulting in high triglyceridemia. Because conversely, the insufficiency enhances hormone-sensitive lipase activity, lipolysis occurs; neutral fat is decomposed in adipose tissues to release FFA and glycerol into the blood, resulting high free fatty acidemia. These results seem to demonstrate that the combined use of the trivalent chromium-nicotinic acid complex and the onion extract enhances insulin sensitivity.

The Zucker fa/fa rats now used had a mutated receptor for leptin as a hormone bringing about the suppression of food intake and the consumption of energy; thus, the action of leptin did not work, resulting in causing polyphagia. The measurements of serum leptin showed no difference between the groups of fa/fa rats; the results of total dietary intake showed that polyphagia could not be suppressed.

What is claimed is:

1. A mixture obtained by mixing trivalent chromium and an onion extract.

2. The mixture according to claim 1 wherein the trivalent chromium is in a form of a trivalent chromium-nicotinic acid complex.

3. A method for improving insulin resistance in mammals comprising administering an effective amount of a mixture of trivalent chromium and an onion extract.

4. The method according to claim 3 wherein the trivalent chromium used is in a form of a trivalent chromium-nicotinic acid complex.

5. A method for suppressing body weight gain in mammals comprising administering an effective amount of trivalent chromium and an onion extract.

6. The method according to claim 5 wherein the trivalent chromium used is in a form of a trivalent chromium-nicotinic acid complex.

7. A method of preventing fatty liver and improving fatty liver in a mammal comprising administering an effective amount of a mixture obtained by mixing trivalent chromium and an onion extract.

8. The method according to claim 7 wherein the trivalent chromium used is in a form of a trivalent chromium-nicotinic acid complex.
